(19) United States
(12) Patent Application Publication
(10) Pub. No.: US 2012/0109092 A1
(43) Pub. Date: May 3, 2012

(54) QUICK-ATTACH SANITARY ABSORBENT ARTICLE ASSEMBLY

(76) Inventors: Jennifer J. Austin, Trenton, NJ (US); Shmuel Dabi, Highland Park, NJ (US); John F. Poccia, Monmouth Beach, NJ (US)

(21) Appl. No.: 13/246,054

(22) Filed: Sep. 27, 2011

Related U.S. Application Data
(60) Provisional application No. 61/409,790, filed on Nov. 3, 2010.

(51) Int. Cl. A61F 13/56

(52) U.S. Cl. 604/385.03

(57) ABSTRACT

A quick-attach absorbent article assembly including a base assembly selectively attachable to a crotch region of an undergarment and a absorbent article selectively attachable to the base assembly. The base assembly is provided with a plurality of protrusions structured and arranged to engage and retain the absorbent article during use.

### CROSS-REFERENCE TO RELATED APPLICATION

This application claims priority to U.S. Provisional Application No. 61/409,790 filed on Nov. 3, 2010.

### FIELD OF THE INVENTION

The present invention relates to a quick-attach absorbent article assembly including a base assembly for attachment to an undergarment and an absorbent article structured to enable a user to quickly attach the absorbent article to the base assembly and easily remove the article from the base assembly when the article is soiled.

### BACKGROUND OF THE INVENTION

Disposable sanitary absorbent articles, such as sanitary napkins, panty liners, incontinence articles, and the like, are well known to those of skill in the art. Typically, such articles include a liquid-permeable body facing cover layer, a liquid-impermeable garment facing barrier layer, and an absorbent core arranged between the cover layer and the barrier layer. The garment facing surface of the barrier layer is typically provided with garment attachment adhesive that enables a user to securely attach the absorbent article to an undergarment during use. Prior to use, the garment attachment adhesive is usually covered with a removable release paper that protects the garment attachment adhesive from contamination prior to use. When ready for use, a user removes the release paper to thereby expose the garment attachment adhesive and places the article in the crotch portion of the user's undergarment so that the garment attachment adhesive secures the article to the crotch portion of the undergarment. Such absorbent articles may also be provided with wings that extend outwardly from a main body of the article. The garment facing surface of each wing is provided with a garment attachment adhesive and the wing is adapted to be folded around an edge of the undergarment and secured by way of the adhesive to an external surface of the undergarment, to thereby hold the article in place.

A problem with absorbent articles having the structure described above is that they can be difficult to apply to the undergarment and remove therefrom. For example, upon removal of the release paper the article may fold upon itself causing two different areas of the article to adhere to one another. Likewise, the wings of such article may fold towards the main body portion and adhere thereto prior to the user correctly positioning the article in the undergarment. Further, it is difficult to handle multiple release papers, especially for those articles including wings. In addition, once the article has been soiled it is difficult to remove the article in a sanitary manner.

In view of the above there is a need for an absorbent article assembly that allows an absorbent article to be easily attached to, and removed from, an undergarment.

### SUMMARY OF THE INVENTION

In view of the foregoing, the present invention provides a quick-attach sanitary absorbent article assembly including a base assembly selectively attachable to an undergarment, the base assembly including a plurality of protrusions, and an absorbent article, wherein the absorbent article is structured and arranged to be engaged by and held in place in the undergarment by the plurality of protrusions.

### BRIEF DESCRIPTION OF THE DRAWINGS

FIG. 1 is a perspective view showing how the absorbent article assembly according to the present invention is installed into a crotch portion of an undergarment;

FIG. 2 is a perspective view similar to FIG. 1 from an underside of the undergarment;

FIG. 3 is a partially exploded view of the base assembly that forms part of the absorbent article assembly according to the present invention;

FIG. 4 is a sectional view taken along line 4-4 in FIG. 3;

FIG. 5 is an exploded view of the absorbent article that forms part of the absorbent article assembly shown in FIG. 1;

FIG. 6 is a sectional view taken along line 6-6 in FIG. 5; and

FIG. 7 is a perspective view showing how a soiled absorbent article is removed from the base assembly and replaced with a fresh absorbent article.

### DETAILED DESCRIPTION OF THE INVENTION

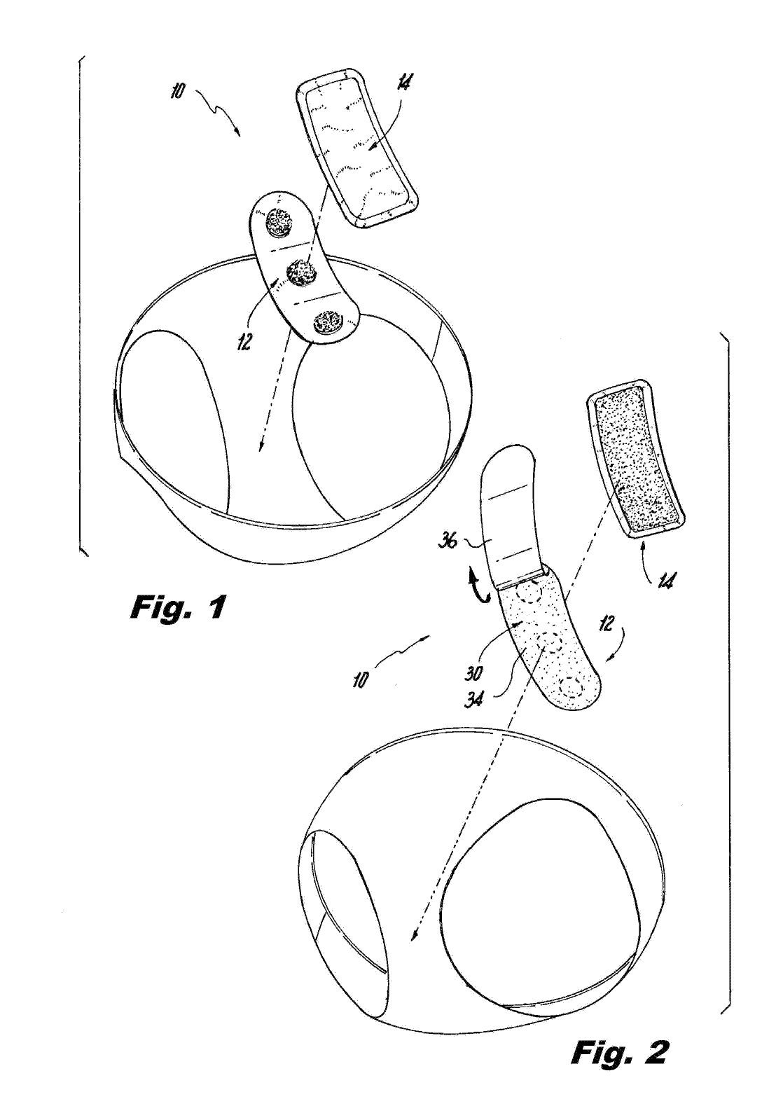

The present invention will be described herein with reference to a sanitary napkin, however the present invention is equally applicable to other sanitary absorbent articles such as panty liners, adult incontinence products and the like. Referring to FIGS. 1 and 2, the quick-attach absorbent article assembly 10 according to the present invention generally includes a base assembly 12 and a sanitary napkin 14.

Referring to FIG. 5, the sanitary napkin 14 includes a liquid-permeable body facing cover layer 16, an attachment layer 22, and an absorbent core 20 arranged between the cover layer 16 and the attachment layer 22. The sanitary napkin 14 may optionally include a liquid-impermeable barrier layer 18 arranged between the absorbent core 20 and the attachment layer 22.

The attachment layer 22 includes an absorbent core facing surface 23 and a base assembly facing surface 24. As will be described in greater detail below, the attachment layer 22 is structured and arranged to engage, and be retained by, the base assembly 12. The sanitary napkin 14 may optionally further include a transfer layer arranged between the cover layer 16 and the absorbent core 20 (not shown in the Figures).

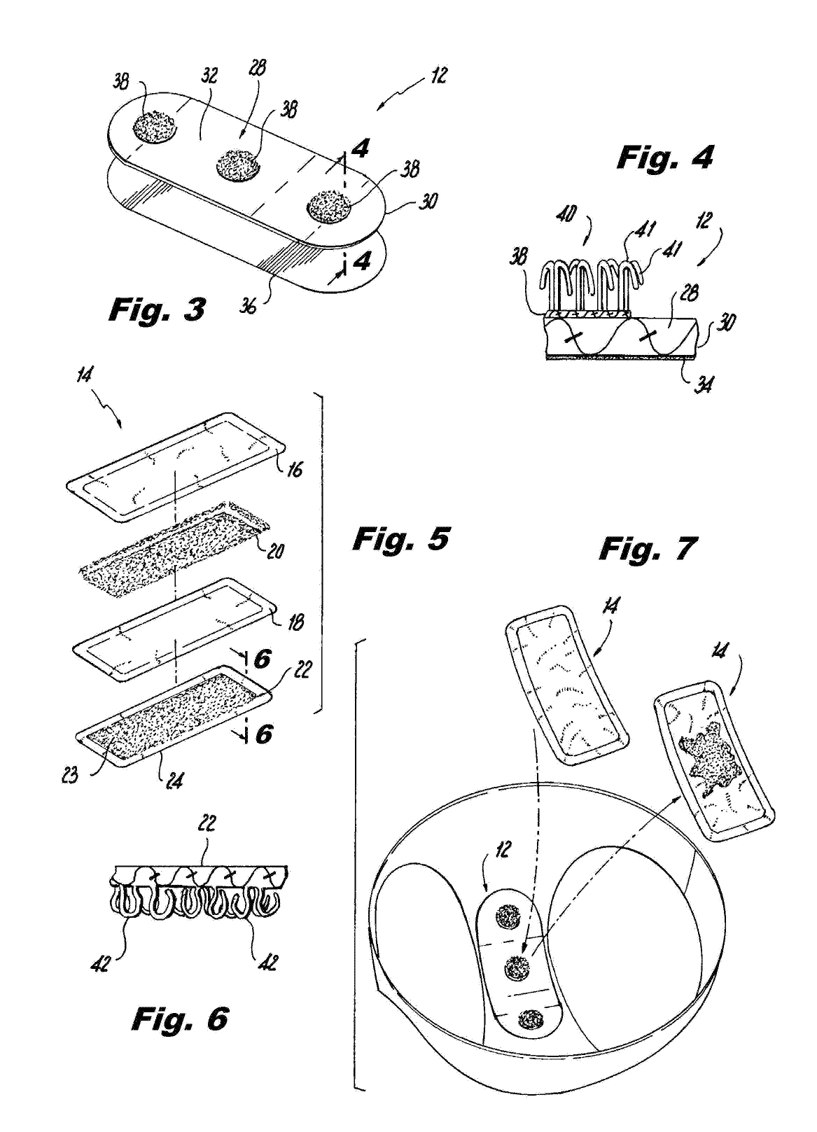

Referring to FIGS. 3 and 4, the base assembly 12 generally includes a base substrate 28 having a garment facing surface 30 and an opposed upper surface 32. As best seen in FIGS. 2 and 4, the garment facing surface 30 is provided with a garment attachment adhesive 34. The garment attachment adhesive 34 is adapted to enable a user to selectively attach the base assembly 12 to an undergarment during use. Prior to use, the garment attachment adhesive 34 may be covered with a removable release paper 36 to protect the adhesive 34 as shown in FIGS. 2 and 3.

The base assembly 12 further includes a fastener substrate 38 that is arranged on top of, and secured to, the upper surface 32 of the base substrate 28. The fastener substrate 38 may be secured to the base substrate 28 by any conventional commercially available construction adhesive, or by any other conventional known means such as heat sealing or the like. As shown in FIG. 4, the fastener substrate 38 includes a plurality of upwardly extending protrusions 40 that are adapted to engage the attachment layer 22 of the sanitary napkin 14 and thereby retain the sanitary napkin 14 securely attached to the base assembly 12 during use. As shown in FIG. 4, in one embodiment of the invention, each protrusion comprises a hook structure 41. The attachment layer 22 of the sanitary napkin 14 is preferably formed from a woven or nonwoven material. As shown in FIG. 6, the attachment layer 22, whether woven or nonwoven, should define a plurality of loops 42 that extend towards the base assembly 12 and are adapted to engage with and be held by the hook structures 41 of the base assembly 12.

As shown in FIG. 3, the fastener substrate 38 may be arranged in a plurality of distinct islands, i.e. each fastener substrate 38 may be arranged in spaced relationship to an adjacent fastener substrate 38. Alternatively, the fastener substrate 38 could be arranged as a continuous layer that extends in an uninterrupted manner.

As described below, the above described structure allows a user to easily attach a sanitary napkin 14 to the base assembly 12, and when the sanitary napkin is soiled, easily remove the soiled sanitary napkin 14 and replace the same with a fresh sanitary napkin 14.

A description of the manner in which the quick-attach absorbent article assembly 10 is used will now be provided. When a user is ready to use the assembly 10 the user first removes the removable release paper 36 from the base assembly 12 to thereby expose the garment attachment adhesive 34, as shown in FIG. 2. Thereafter, the user manually places the base assembly 12 in the crotch portion of the user's undergarment so that the garment facing surface 30 of the base assembly 12 is facing the user's undergarment. By pressing the base assembly 12 against the crotch portion of the undergarment the user may securely attach the base assembly 12 thereto by means of the garment attachment adhesive 34. Once the base assembly 12 is in place the user then arranges the absorbent article 14 on top of the base assembly 12 such that the base assembly facing surface 24 of the attachment layer 22 faces the base assembly 12. By pressing firmly down on the article 14 the protrusions 40 of the base assembly 12 engage the attachment layer 22 and thereby function to hold the article 14 in place for use. Once the article 14 has been soiled the user may manually remove the article 14 and replace the same with a new fresh article 14. During this replacement process the base assembly 12 stays in place in the crotch portion of the undergarment thereby significantly simplifying the change experience of the user. It is important that the garment attachment adhesive 34 be selected so that the effective removal force required to remove the base assembly 12 from the undergarment is greater than the removal force required to remove the sanitary napkin 14 from the base assembly 12. This will insure that the base assembly 12 will stay in place in the undergarment when the article 14 is removed from the base assembly 12. The base assembly 12 is removable from the undergarment prior to laundering.

### Cover Layer

The liquid-permeable body facing cover layer 16 may be a relatively low density, bulky, high-loft non-woven web material. The cover layer 16 may be composed of only one type of fiber, such as polyester or polypropylene or it may include a mixture of more than one fiber. The cover may be composed of bi-component or conjugate fibers having a low melting point component and a high melting point component. The fibers may be selected from a variety of natural and synthetic materials such as nylon, polyester, rayon (in combination with other fibers), cotton, acrylic fiber and the like and combinations thereof.

The cover layer 16 preferably has a relatively high degree of wettability, although the individual fibers comprising the cover may not be particularly hydrophilic. The cover material should also contain a great number of relatively large pores. This is because the cover layer 16 is intended to take-up body fluid rapidly then transport it away from the body and the point of deposition. Therefore, the cover layer contributes little to the time taken for the napkin to absorb a given quantity of liquid (penetration time).

Advantageously, the fibers which make up the cover layer 16 should not lose their physical properties when they are wetted, in other words they should not collapse or lose their resiliency when subjected to water or body fluid. The cover layer 16 may be treated to allow fluid to pass through it readily. The cover layer 16 also functions to transfer the fluid quickly to the underlying layers of the napkin. Thus, the cover layer 16 is advantageously wettable, hydrophilic and porous. When composed of synthetic hydrophobic fibers such as polyester or bi-component fibers, the cover layer 16 may be treated with a surfactant to impart the desired degree of wettability.

Alternatively, the cover layer 16 can also be made of polymer film having large pores. Because of such high porosity, the film accomplishes the function of quickly transferring body fluid to the inner layers of the underlying absorbent layers.

The cover layer 16 may be attached to the underlying layers of the sanitary napkin 14 by adhesion and/or other suitable means know to those of skill in the art.

In one embodiment of the invention the cover layer comprises a hot through air bonded nonwoven composed of PE/PET bicomponent fibers, such nonwoven being commercially available from Shalag Industries, Ltd., Upper Galilee, Israel.

### Transfer Layer

The optional transfer layer (not shown in the Figures) may be composed of fibrous materials, such as wood pulp, polyester, rayon, flexible foam, or the like, or combinations thereof. The transfer layer may also optionally include a superabsorbent polymer (SAP) material. The transfer layer may also comprise thermoplastic fibers for the purpose of stabilizing the layer and maintaining its structural integrity. The transfer layer may be treated with surfactant on one or both sides in order to increase its wettability, although generally the transfer layer is relatively hydrophilic and may not require treatment. The transfer layer is preferably bonded on both sides to the adjacent layers, i.e. to the cover layer 16 and the underlying core 20.

In one embodiment of the invention the transfer layer comprises an airlaid pulp material commercially available from P.H. Glatfelter Company, York, Pa., under product code MH090.102.

### Absorbent Core

The absorbent core 20 may comprise a single layer of material or may comprise multiple layers. In one embodiment, the core 20 is a blend or mixture of cellulosic fibers and superabsorbent disposed therein. Cellulosic fibers that can be used in the second absorbent layer 20 are well known in the art and include wood pulp, cotton, flax and peat moss. Wood pulp is preferred. Pulps can be obtained from mechanical or chemi-mechanical, sulfite, kraft, pulping reject materials, organic solvent pulps, etc. Both softwood and hardwood species are useful. Softwood pulps are preferred. It is not necessary to treat cellulosic fibers with chemical debonding agents, cross-linking agents and the like for use in the present material. Some portion of the pulp may be chemically treated as discussed in U.S. Pat. No. 5,916,670 to improve flexibility of the product, the content of which is incorporated by reference in its entirety. Flexibility of the material may also be improved by mechanically working the material or tenderizing the material.

The core 20 can contain any superabsorbent polymer (SAP) which are well known in the art. For the purposes of the present invention, the term “superabsorbent polymer” (or “SAP”) refers to materials which are capable of absorbing and retaining at least about 10 times their weight in body fluids under a 0.5 psi pressure. The superabsorbent polymer particles of the invention may be inorganic or organic crosslinked hydrophilic polymers, such as polyvinyl alcohols, polyethylene oxides, crosslinked starches, guar gum, xanthan gum, and the like. The particles may be in the form of a powder, grains, granules, or fibers. Preferred superabsorbent polymer particles for use in the present invention are crosslinked polyacrylates, such as the product offered by Sumitomo Seika Chemicals Co., Ltd. Of Osaka, Japan, under the designation of SA70N and products offered by Stockhausen Inc.

In a specific example, the core 20 is a material containing from 90% to about 40% percent cellulosic fiber, about 10% to about 60% SAP. The core 20 may comprise a material manufactured by using air-laying means well known in the art.

In one embodiment of the invention the core 20 is relatively thin, high swelling absorbent material sold under the trade name NOVATHIN® INT-002 available from EAM Corporation located in Jessup, Ga., U.S.A.

The absorbent core 20 may be adhered to the adjacent layers, e.g. the cover layer 16 (or transfer layer if such layer is employed) and the barrier layer 18 by means of a conventional construction adhesive well known to those of skill in the art.

### Barrier Layer

Underlying absorbent core is the liquid-impermeable barrier layer 18. The barrier layer 18 preferably comprises liquid-impervious film material so as to prevent liquid that is entrapped in the absorbent core 20 from egressing the sanitary napkin 14 and staining the wearer's undergarment. The barrier layer 18 is preferably made of polymeric film, although it may be made of liquid-impervious, air-permeable material such as repellent-treated non-woven or micropore films or foams.

The barrier layer 18 may be breathable, i.e., permits vapor to transpire. Known materials for this purpose include nonwoven materials and microporous films in which microporosity is created by, inter alia, stretching an oriented film. Single or multiple layers of permeable films, fabrics, melt-blown materials, and combinations thereof that provide a tortuous path, and/or whose surface characteristics provide a liquid surface repellent to the penetration of liquids may also be used to provide a breathable backsheet. The cover layer 16 and the barrier layer 18 are preferably joined along their marginal portions so as to form an enclosure or flange seal that maintains the absorbent core 20 captive. The joint may be made by means of adhesives, heat-bonding, ultrasonic bonding, radio frequency sealing, mechanical crimping, and the like and combinations thereof.

In one specific embodiment of the invention the barrier layer 18 comprises a polyethylene film material of the type commercially available from Berry Plastics Corporation, Evansville, Ind.

### Attachment Layer

The attachment layer 22 is preferably formed from a woven or nonwoven material. Nonwoven materials may include spunmelts, thermo-bonds, through air bonds, needlepunch or spunlace materials. Alternatively the attachment layer may be formed from polymeric films provided that such films are provided with a sufficient structure to engage with and be retained by the protrusions 40 of the base assembly 12. For example such films may be provided with holes, fibrillated, or provided with some other three dimensional structure that enables the film to engage with and be retained by the protrusions 40.

As shown in FIG. 6, the attachment layer 22 in one embodiment of the invention defines a plurality of loops 42 that extend towards the base assembly 12 and are adapted to engage with and be held by the hook structures 41 of the base assembly 12. In this manner, the loops 42 and hook structures 41 function to hold the sanitary napkin 14 in place during use and also permit the user to manually remove the napkin 14 once the napkin 14 has been soiled. The attachment layer 22 may comprise a single layer or may comprise a multi-layer laminate provided that such laminate includes a layer that faces the base assembly having a plurality of loops 42 as described herein. The attachment layer 22 is preferably secured to the barrier layer 18, or core structure if such barrier 18 is omitted, by means of a conventional construction adhesive well known to those of skill in the art.

In one specific embodiment of the invention the attachment layer 22 comprises a liquid-impermeable, air permeable, laminate structure including a microporous film core facing layer that is bonded to a spunbond nonwoven base assembly facing layer. Since the attachment layer 22 is liquid-impermeable the barrier 18 may be omitted if desired. In a specific embodiment of the invention the attachment layer 22 comprises a 35 gsm laminate structure of this type commercially available from Kang Na Hsiung Enterprises Co., Ltd., Taipei, Taiwan under product code CL035-CPS5-N0.

### Base Substrate

The base substrate 28 may be formed from any conformable material such as a suitably flexible woven or nonwoven material that will move with the user's undergarment. The base substrate 28 may be formed of a single layer or may be formed from a multi-layer laminate. The base substrate 28 may optionally be formed from liquid-impermeable materials or may be rendered liquid-impermeable by a separate treatment. As discussed above, the garment facing surface 30 of the base substrate 28 is provided with a garment attachment adhesive 34.

The garment attachment adhesive 34 may be any suitable pressure-sensitive adhesive. As used herein, the term pressure-sensitive adhesive refers to any releasable adhesive or releasable tenacious means. Suitable adhesive compositions, include, for example, water-based pressure-sensitive adhesives such as acrylate adhesives. Alternatively, the adhesive composition may include adhesives based on the following: emulsion or solvent-borne adhesives of natural or synthetic polyisoprene, styrene-butadiene, or polyacrylate, vinyl acetate copolymer or combinations thereof; hot melt adhesives based on acrylics or suitable block copoylmers—suitable block copolymers for use in the invention include linear or radial co-polymer structures having the formula (A-B)x wherein block A is a polyvinylarene block, block B is a poly(monoalkenyl) block, x denotes the number of polymeric arms, and wherein x is an integer greater than or equal to one. Suitable block A polyvinylarenes include, but are not limited to Polystyrene, Polyalpha-methylstyrene, Polyvinyltoluene, and combinations thereof. Suitable Block B poly(monoalkenyl) blocks include, but are not limited to conjugated diene elastomers such as for example polybutadiene or polyisoprene or hydrogenated elastomers such as ethylene butylene or ethylene propylene or polyisobutylene, or combinations thereof; hot melt adhesives based upon acrylic polymers. Commercial examples of these types of block copolymers include Kraton™ elastomers from Shell Chemical Company, Vector™ elastomers from Dexco, Solprene™ from Enichem Elastomers and Stereon™ from Firestone Tire & Rubber Co.; hot melt adhesive based on olefin polymers and copolymers where in the olefin polymer is a terpolymer of ethylene and a co-monomers, such as vinyl acetate, acrylic acid, methacrylic acid, ethyl acrylate, methyl acrylate, n-butyl acrylate vinyl silane or maleic anhydride. Commercial examples of these types of polymers include Ateva™ (polymers from AT plastics), Nucrel™ (polymers from DuPont), Escor™ (from Exxon Chemical). In a specific embodiment of the present invention the garment attachment adhesive is an acrylic hot melt Duro-Tak 34-546B from Henkel AG & Co. KGaA, Dusseldorf, Germany.

In one specific embodiment of the invention, the base substrate 28 comprises a liquid-impermeable, air permeable, laminate structure including a microporous film garment facing layer that is bonded to a spunbond nonwoven fastener substrate facing layer. Laminate structures of this type are commonly used as a backsheet material for diapers. In a specific embodiment of the invention the attachment layer 22 comprise a 35 gsm laminate structure of this type commercially available from Kang Na Hsiung Enterprises Co., Ltd., Taipei, Taiwan under product code CL035-CPS5-N0.

### Fastener Substrate

The fastener substrate 38 is preferably formed from a polymeric material including a plurality of upwardly extending protrusions 40 as described above. In one embodiment of the invention each protrusion comprises a hook structure 41. Polymeric materials of this type are commercially available from Velcro USA, Inc., Manchester, N.H.

In one specific embodiment of the invention the fastener substrate 38 comprises a polymeric material commercially available from Velcro USA, Inc., Manchester, N.H. under product code HTH 847.

The fastener substrate 38 is bonded to the article facing surface of the base substrate 28 by any conventional known means in the art such as adhesion or ultrasonic bonding.

### Example

A quick-attach absorbent article assembly according to one embodiment of the invention was constructed as follows:

### Sanitary Napkin

Cover:
HTA 27 gsm bico fibers PE/PET, STA4ETW27, Shalag Industries, Ltd, Upper Galilee, Israel.

Adhesive layer:
NW1023 1.0 gsm (100% coverage) stryrenated block copolymer based hot melt pressure sensitive adhesive, HB Fuller, St Paul, Minn.

Transfer layer:
90 gsm airlaid 90% pulp, MH090.102, P.H. Glatfelter Company, York, Pa.

Adhesive layer:
NW1023 4.0 gsm (100% coverage,) stryrenated block copolymer based hot melt pressure sensitive adhesive, HB Fuller, St Paul, Minn.

Core:
Novathin 208 gsm 25% SAP, 56 mm×205 mm, Novathin INT-002, EAM corporation, Jesup, Ga.

Adhesive layer:
NW1023 4.0 gsm (100% coverage,) stryrenated block copolymer based hot melt pressure sensitive adhesive, HB Fuller, St Paul, Minn.

Barrier:
Pliant, 0.8 mil PE metallocene, Berry Plastics Corporation, Evansville, Ind.

Adhesive layer:
NW1042 22.0 gsm (−40% coverage,) stryrenated block copolymer based hot melt pressure sensitive adhesive, HB Fuller, St Paul, Minn.

Attachment layer:
a 35 gsm laminate structure of this type commercially available from Kang Na Hsiung Enterprises Co., Ltd., Taipei, Taiwan under product code CL035-CPS5-N0.

### Base Assembly

Base substrate:
a 35 gsm laminate structure of this type commercially available from Kang Na Hsiung Enterprises Co., Ltd., Taipei, Taiwan under product code CL035-CPS5-N0.

Garment attachment adhesive on a garment facing surface of the base substrate:
an acrylic hot melt Duro-Tak 34-546B from Henkel AG & Co. KGaA, Dusseldorf, Germany.

Fastener substrate:
a polymeric material commercially available from Velcro USA, Inc., Manchester, N.H. under product code HTH 847.

Adhesive between base substrate and fastener substrate:
NW1042 22.0 gsm stryrenated block copolymer based hot melt pressure sensitive adhesive, HB Fuller, St Paul, Minn.

While particular embodiments of the preset invention have been illustrated and described, it would be obvious to those skilled in the art that various other changes and modifications can be made without departing from the spirit and scope of the invention. It is therefore intended to cover in the appended claims all such changes and modifications that are within the scope of the invention.

We claim:

1. A quick-attach absorbent article assembly comprising:
a base assembly selectively attachable to an undergarment, said base assembly including a plurality of protrusions; and
an absorbent article;
wherein said absorbent article is structured and arranged to be engaged by and held in place in said undergarment by said protrusions.

2. The quick-attach absorbent article assembly according to claim 1, wherein each of said protrusions comprises a hook structure.

3. The quick-attach absorbent article assembly according to claim 2, wherein said base assembly comprises a base substrate and a fastener substrate.

4. The quick-attach absorbent assembly according to claim 3, wherein said fastener substrate includes a plurality of said hook structures.

5. The quick-attach absorbent article assembly according to claim 4, wherein a garment facing surface of said base substrate includes a garment attachment adhesive adapted to allow a user to selectively attach the base assembly to said undergarment.

6. The quick-attach absorbent article assembly according to claim 5, wherein said absorbent article comprises an attachment layer.

7. The quick-attach absorbent article assembly according to claim 6, wherein said attachment layer includes a plurality of loops, said loops being structured and arranged to be engaged and retained by said hook structures.

8. The quick-attach absorbent article assembly according to claim 7, wherein said absorbent article further comprises a liquid-permeable body facing cover layer.

9. The quick-attach absorbent article assembly according to claim 8, wherein said absorbent article further comprises an absorbent core arranged between said cover layer and said attachment layer.
